(19) United States
(12) Patent Application Publication
(10) Pub. No.: US 2004/0098070 A1
(43) Pub. Date: May 20, 2004

(54) Apparatus for real time measure/control of intra-operative effects during laser thermal treatments using light scattering

(76) Inventors: Stuart Mohr, Redwood City, CA (US); William Telfair, San Jose, CA (US); Bradley Renton, Pleasanton, CA (US); Eduardo Arias, Los Altos Hills, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/666,536

(22) Filed: Sep. 19, 2003

Related U.S. Application Data
(60) Provisional application No. 60/412,465, filed on Sep. 20, 2002.
(60) Provisional application No. 60/473,968, filed on May 28, 2003.

(51) Int. Cl. A61N 1/00

(52) U.S. Cl. 607/89

(57) ABSTRACT

A treatment apparatus for a tissue site includes a scattered light measurement device. The scattered light measurement device produces an excitation beam to scatter from the tissue site and monitors temperature dependent changes at the tissue site. An output device produces an output to an observer that is indicative of the temperature change at the tissue site. The output device can produce a variety of different outputs including but not limited an output through a computer, through a heads up display, through a slit lamp, an audible output or a print out of information.

### CROSS-REFERENCE TO RELATED APPLICATIONS

This application claims the benefit of Ser. Nos. 60/412,465 filed Sep. 20, 2002 and 60/473,968 filed May 28, 2003, both of which applications are incorporated herein in their entirety.

### BACKGROUND OF THE INVENTION

1. Field of the Invention

This invention relates generally to methods and apparatus for monitoring thermal effects in the body, and more particularly to methods and apparatus for monitoring thermal effects on the retina during thermal treatment.

2. Description of Related Art

There are several diseases that can cause severe visual impairment, which can be treated with a laser. Some of these include glaucoma, diabetic retinopathy, macular edema, central serous retinopathy and age-related macular degeneration (AMD). AMD represents the major cause of severe vision loss (SVL) of people in the United States between the ages of 65 and 80. The incidence of AMD in the United States alone is currently estimated at 2 million new cases per year. A widely used form of treatment for these disorders is laser photocoagulation (LPC)

Laser treatment, and in particular LPC, has become the standard of care for a number of retinal and choroidal diseases and pathologies. More recently it has been expanded to lower dose treatments and there is a trend toward earlier treatment made possible by Minimum Intensity Photocoagulation (MIP) treatments.

Conventional LPC is a photothermal process that relies on visible endpoints to the user. These visible endpoints are intensely treated regions in the retina where temperature elevations of 60° C. or higher are experienced and the retina has bleached, irreversibly losing it's normal transparency. The retina is transparent to most laser wavelengths so chromophores, that absorb the light energy and converted it to heat, primarily absorb laser energy. The main absorbing chromophores are melanin in the RPE and hemoglobin in the retinal and choroidal blood vessels. The retina is heated by thermal conduction from these absorbing structures that are primarily located beneath the retina. This means that when a laser treatment becomes visible, there is already a full thickness burn below the retina with irreversible changes to the RPE and damage to the Retina. These burns result in immediate vision loss at the treatment location. Multiple laser treatments, such as Pan-Retinal Photo Coagulation used for Diabetic Retinopathy, results in several lines of vision loss.

MIP is a term given to treatments where a minimum amount of laser energy is delivered to produce a desired endpoint while minimizing collateral damage. In some cases using pulse regimes to create thermal confinement to a treatment location and limit the extent of damage performs this. In other treatments less laser power is used resulting in smaller thermal gradients and preservation of retinal function. Research in MIP has shown that for many cases of AMD it is not necessary to create a full thickness burn to produce clinical effects. It has been demonstrated that a temperature elevation above a clinical affectivity threshold, yet below the damage threshold, will produce a photothermal, photochemical, photomechanical, and/or biostimulation effect that halts the progression of the disease equivalent to conventional LPC but unlike conventional LPC, does not adversely affect vision. One such procedure that uses this technique is Transpupillary Thermal Therapy (TTT). TTT uses a long low irradiance pulse to minimize the increase in temperature of the retina. When the temperature of the retina is increased roughly 10 degrees Celsius and is maintained for approximately one minute studies show that, in a significant number of treated eyes, natural progression of the disease is halted and vision is preserved. Due to the limited temperature elevation, photoreceptors and ganglion cells are preserved. This allows treatment anywhere on the retina, including over the fovea. Using conventional LPC treatments cannot be performed over the fovea due to the risk of SVL caused by the treatment.

The difficulty with this treatment is the necessity to maintain a temperature delta in the eye capable of producing clinically effective results but small enough to avoid damage to the retina. Too little temperature elevation results in a non-treatment and too much elevation results in a full thickness burn and vision loss. Variation in pigmentation, size and number of choroidal neovascular networks (CNV), sub retinal fluid, etc. from patient to patient results in different required treatment parameters to achieve the optimal thermal effect. Doctors currently use a complex set of variables to aid them in determining a safe, yet effective, treatment dose.

Earlier intervention, and stabilization of vision, offers the possibility of providing patients with better-sustained end vision because their vision stabilizes while it is still good. MIP has been slow in clinical acceptance due to the difficulties that face a doctor when attempting to perform a treatment without visual cues and the possible risks of over-treating and damaging vision in a patient that is just beginning to show vision loss. As a result, conventional LPC is performed to halt the progression of the disease but not until late stages of the disease when vision losses associated with the treatment itself are less significant than those caused by the disease and the likelihood of continued visual loss due to the disease is high.

Light scattering has been shown to have a significant scatter intensity change in hemoglobin at the hemoglobin melting point by Protein Solutions, Inc. The melting point of hemoglobin occurs between 42° C. and 47° C. This is also the temperature where apoptosis and early stages of necrosis in the RPE and vascular endothelium occur but is still within temperatures that are acceptable to the overlying neurosensory retina. Static light scattering intensity increases exponentially through the temperature range providing potential feedback to the intensity of the treatment. Detection of temperature levels around which apoptosis occurs, is of interest because up regulation of gene expressions, which occur as a result of apoptosis, is one hypothesis as to why MIP procedures result in stabilization of vision. Scattering intensity changes as a result of proteins denaturing. Different proteins denature at different temperatures. This allows a system to monitor changes in the proteins/tissues that are desired and to tailor a treatment that damages only the target or protects more sensitive structures.

Polarization retention has been shown as an additional method of monitoring tissue in biologic structures. It has been shown that the degree of polarization changes as a function of temperature in blood, arteries, and fat. As temperature increases the degree of polarization retention increases. At 35° C. polarization sensitivity has been measured as ˜0.3 of incident polarization. At 45° C. the degree of polarization sensitivity approaches 0.8. This relationship between temperature and polarization retention has been proposed to assist in imaging various cancers. An alternative usage of the change in polarization retention would be to determine the degree of temperature change affecting the backscattered light. (Polarized Light Imaging Through Biologic Tissue, Vanitha Sankaran and Duncan Maitland, UC Davis & Lawrence Livermore)

Birefringence of light in liquid crystals is dependant on applied voltage, wavelength, and temperature. Depending on the crystalline structure, the effect of temperature can be significant. In the case of pentyl-cyanobiphenyl (5CB) the birefringence was about 0.17 at 27° C. and 0.12 at 35° C. (Nick Oullette and Lisa Larrimore) In the application of thermal treatments in biologic tissues, there is no applied voltage; the wavelength for monitoring changes is held constant and or known, leaving temperature as the dependant variable. Scattered or returned light from the birefringent structure should change as a function of tissue temperature. A system capable of monitoring changes in polarization and phase sensitivity could be used to track these changes. Birefringence has also been shown to change in collagen when it is thermally damaged by laser irradiation (Two-dimensional birefringence imaging in biological tissue by polarization-sensitive optical coherence tomography. Johannes F. de Boer, Thomas E. Milner, Martin J. C. van Gemert, J. Stuart Nelson. Optics Letters Vol. 22, No. 12 Jun. 15, 1997). This effect should also be apparent in other birefringent structures in the eye such as Henle's layer located at the macula. A detection system capable of monitoring minute changes in birefringence such as the GDx system from Laser Diagnostics Technologies could provide a more sensitive method of visualizing the retina and allow a user to halt treatment before the eye is significantly damaged.

Phase Sensitive OCT (PS-OCT) can be used to monitor opto-acoustic signals. This could be used to monitor opto-acoustic signals from the eye that would indicate retinal temperature during treatment. PS-OCT is one commercially available method of monitoring birefringence and polarization related changes in the eye. Another system, which is capable of monitoring polarization and birefringence measurements in the eye, is a form of scanning laser ophthalmoscope. Either system can be used to detect differences in static light scattering at various polarization angles relative to the incident light. These commercially available systems are not the only systems capable of performing these measurements. Any combination of these technologies would allow for potential additional data, which would assist in determining temperature related changes in the treatment region.

There is a need for a new apparatus capable of monitoring sub-visible-threshold effects at a tissue site, particularly the retina during laser photocoagulation, and a laser delivery system capable of dynamically adjusting treatment parameters to consistently deliver therapeutically effective treatments limiting iatrogenic damage. There is a further need for a laser system that allows a pre-programmed treatment history/profile to be entered, and a monitoring device capable of detecting and allowing real-time laser adjustment, either manually or automatically. There is yet a another need for a laser system that provides for real time laser adjustment, maintains a time/temperature history, enable physicians to treat multiple diseases of the eye, regardless of location, at an earlier stage resulting in better preserved vision, with little to no risk of causing visual impairment during the treatment.

### SUMMARY OF THE INVENTION

Accordingly, an object of the present invention is to provide an apparatus, and its methods of use, for treating a tissue site as well as having a visible endpoint for treatment.

Another object of the present invention is to provide an apparatus, and its methods of use, that is capable of non-invasively monitoring real time temperature effects at a tissue site and to ensure that the desired treatment has been performed.

Yet another object of the present invention is to provide an apparatus, and its methods of use, that non-invasively monitors real time parameter effects on the retina at the location of the treatment, to prevent damage to the retina, and ensure that the desired treatment has been performed.

A further object of the present invention is to provide an apparatus, and its methods of use, directed to offering a solution to the challenges affecting MIP and specifically to the problem that there is no visible endpoint

Still another object of the present invention is to provide an apparatus, and its methods of use, that enables visualization changes in the retina that are caused by the application of laser irradiation, and the subsequent photothermal, photochemical, and or photomechanical processes.

Another object of the present invention is to provide an apparatus, and its methods of use, that monitors changes in hemoglobin or other structures in the retina and offers a treatment-induced threshold.

Yet another object of the present invention is to provide an apparatus, and its methods of use, with a treatment threshold measured by monitoring changes in light scattering intensity caused by thermal elevation.

Still another object of the present invention is to provide an apparatus, and its methods of use, that includes a monitoring device capable of providing treatment information to the physician by audio, visual, or printed form.

Still a further object of the present invention is to provide an apparatus, and its methods of use, that includes a monitoring device used to provide information used to increase or decrease laser parameters, provide warning signals to inform the user that a threshold is being approached or passed, provide up to date information related to the treatment at that point in time allowing the doctor to make informed changes to the treatment.

Another object of the present invention is to provide an apparatus, and its methods of use, that allows the user to enter predetermined treatment parameters and goals into a system that has the ability to control energy parameters to achieve and maintain a predetermined temperature history profile by actively adjusting the pulse duration, power, frequency, and or irradiance.

These and other objects of the present invention can be achieved in a treatment apparatus for a tissue site. A scattered light measurement device produces an excitation beam to scatter from the tissue site and monitor, temperature dependent changes at the tissue site. An output device produces an output to an observer that is indicative of the temperature change at the tissue site. The output device can produce a variety of different outputs including but not limited an output through a computer, with a heads up display, through a slit lamp, an audible output or a print out of information.

In another embodiment of the present invention, a treatment apparatus for a tissue site includes a scattered light measurement device that produces an excitation beam to scatter from the tissue site and monitor, temperature induced changes at the tissue site. An output device produces an output to an observer that is indicative of the temperature induced changes at the tissue site. The output device can produce a variety of different outputs including but not limited an output through a computer, with a heads up display, through a slit lamp, an audible output or a print out of information.

In another embodiment of the present invention, a treatment apparatus for a tissue site, includes an energy device that produces energy delivered to the tissue site. A scattered light measurement device delivers an excitation beam to scatter off the tissue site and monitor temperature dependent changes of the tissue site. A control device is coupled to the energy device and the light scattering measurement device. In response to a measurement from the light scattering measurement device, the control device controls the output energy of the treatment beam while the scattered light measurement device monitors the temperature dependent changes of the tissue site.

In another embodiment of the present invention, a treatment apparatus for a tissue site includes an energy device that produces energy delivered to the tissue site. A scattered light measurement device delivers an excitation beam to scatter off the tissue site and monitors the scattered light. A control device is coupled to the energy device and the scattered light measurement device. In response to a temperature change, or a change of baseline temperature of the tissue site, the control device controls the output energy of the treatment beam to the tissue site.

In another embodiment of the present invention, a treatment apparatus for an eye includes an energy device that produces a treatment beam delivered to a tissue site. A scattered light measurement device delivers an excitation beam to scatter off the treatment eye. A control device is coupled to the light energy device and the scattered light measurement device. In response to a change in the scattered light from the excitation beam, the control device controls the output energy of the treatment beam while the scattered light measurement device monitors the change in scatter light.

In another embodiment of the present invention, a method of treatment at a tissue site provides an apparatus for monitoring a temperature change at the tissue site. The apparatus includes a scattered light measurement device, which produces an excitation beam, and an output device. An excitation beam is produced and scatters from the tissue site. Temperature dependent changes of the tissue site are monitored. An indication of the temperature change at the tissue site is provided to an observer.

In another embodiment of the present invention, a method of treatment at a tissue site provides an apparatus for monitoring a temperature induced change at the tissue site. The apparatus includes a scattered light measurement device, which produces an excitation beam, and an output device. An excitation beam is produced and scatters from the tissue site. The temperature induced changes of the tissue site are monitored. An indicative of the temperature induced change at the tissue site is provided to an observer.

In another embodiment of the present invention, the treatment apparatus includes an energy device that produces energy delivered to the tissue site. A scattered light measurement device delivers an excitation beam to scatter off the tissue site and monitor temperature dependent changes of the tissue site. A control device is coupled to the energy device and the scattered light measurement device. In response to a measurement from the scattered light measurement device, the control device controls the output energy of the treatment beam while the scattered light measurement device monitors the temperature dependent changes of the eye.

In another embodiment, in response to a temperature change or a change of baseline temperature of the tissue site, the control device controls the output energy of the treatment beam to the tissue site. In various embodiments the scattered light correlates to a birefringence effect resulting from the delivery of the treatment beam to the tissue site, to a chemical effect resulting from the delivery of the treatment beam to the tissue site, to a thermal effect resulting from the delivery of the treatment beam to the tissue site, to a mechanical effect resulting from the delivery of the treatment beam to the tissue site, and the like. The scattered light can be specular and/or diffuse scattered light.

In another embodiment, in response to a change in the scattered light from the excitation beam, the control device controls the output energy of the treatment beam while the scattered light measurement device monitors the change in scatter light. The treatment can deliver the treatment beam to the tissue site until a threshold is reached.

In one specific embodiment, the energy device is a light source, such as a laser, and the tissue site is an eye, such as a retina of the eye.

In various embodiments, the apparatus of the present invention may contain multiple energy sources both for treatment and monitoring in which any or all parameters, including but not limited to, power, energy, irradiance, duration, temperature profile, number of pulses, and the like, can be individually pre-programmed and adjusted to produce the desired treatment effect. Each function can be designed to gradually produce the intended therapeutic photothermal, photomechanical and/or photochemical effect or to halt or change a treatment at any predetermined condition. The treatment device parameters can be adjusted according to input from the monitoring apparatus to maintain an optimum effect for the desired treatment.

More specifically the apparatus of the present invention can include a monitoring system incorporated into a laser delivery system capable of monitoring real time temperature related effects on proteins in the body and providing feedback control to the operator, or directly to the system itself. This feedback provides real-time-treatment effect data enabling either operator control, or automatic control, of the laser parameters to maintain a preprogrammed temperature profile and history by.

A variety of scattered light measurement devices can be utilized, including but not limited to a, polarization device, phase sensitive optical device, a birefringent device and the like. The phase sensitive optical device can be a phase sensitive optical coherence tomographer (PS-OCT). The polarization device can be a scanning laser ophthalmoscope or a polarization sensitive device. The PS-OCT observes phase sensitive changes or changes in polarization at specific depths within the tissue site. The polarization device can monitor depth specific changes in the tissue site and/or full thickness changes in the tissue site. In one embodiment, the scattered light measurement device provides measurements at the tissue site and at an off tissue site. In one embodiment, the scattered light measurement device provides measurement by comparing a current measurement to a baseline measurement at the tissue site. The scattered light measurement device can provide measurement at the treatment location and at an off tissue site and determines a change at the tissue site by comparing the off tissue site with the tissue site. The scattered light measurement device can measure absolute temperature.

### BRIEF DESCRIPTION OF THE DRAWINGS

FIG. 1 is a block diagram illustrating one embodiment of a treatment apparatus for a tissue site. A scattered light measurement device produces an excitation beam to scatter from the tissue site and monitor, temperature dependent changes or temperature induced changes at the treat site. An output device produces an output to an observer that is indicative of the temperature change, or the temperature induced change at the tissue site. The output device can produce a variety of different outputs including but not limited an output through a computer, through a slit lamp, an audible output or a print out of information.

FIG. 2 is an optical schematic illustrating one embodiment of a treatment apparatus for a tissue site. A scattered light measurement device is composed of a scatter source and a detector. The scatter source produces a polarized excitation beam to scatter from the tissue site and the detector monitors scattered light returned through a polarizer to monitor temperature dependent changes or temperature induced changes at the treat site. This scattered light measurement device is co-aligned with the treatment laser, with the view of the physician/user and the white light illumination source.

### DETAILED DESCRIPTION OF THE INVENTION

The user (10) has the ultimate control of the delivery of energy to the tissue sit. Depending on the histology and structure of the eye the doctor, or user, can enter parameters for the treatment (11). These parameters can control any of the functions of the laser. These include power, pulse duration, and pulse interval. In addition they can include desired treatment modalities such as desired temperature/temperature effect history profiles, desired time at a specified temperature elevation, temperature rise time, and temperature fall time. The user may also have the ability to determine the level of automatic control the laser system provides.

One control that the user has is the ability to start (12) and stop laser delivery (13) at any point in the treatment. The laser system is controlled by a footswitch or other manually actuated device requiring user interaction at all times. The user is continuously monitoring the eye for visual information and by releasing the footswitch, or equivalent device used to actuate the laser, can immediately halt the progression of the treatment regardless of history.

To aid the user in visualizing the eye there can be several forms of feedback. Visual feedback (15) in the form of a light or a display can signal to the doctor the level of treatment provided and provide additional feedback indicating the need to increase or decrease power as well as information related any or all of the following: actual temperature, treatment history, temperature profile of the treatment, pulse duration, or time at given temperature. This same information could be portrayed to the user through Audio signal (16) such as a beep or voice commands or through printed feedback (17). Visualization of the treatment eye (50) can be obtained by using a slit lamp or other direct viewing system. In addition, non-direct visualization and visible feedback could be provided by other means such as a video/monitoring system where treatment information is updated real time on a monitoring device.

In one specific embodiment, the energy device (20) is an 808+/−5 nm infrared laser (22). The wavelength can be virtually any wavelength provided it has sufficient transmission efficiency to pass through the cornea, lens and aqueous. This can include visible wavelengths as well as wavelengths further into the infrared. The desired endpoint is to non-invasively cause general heating of the retina. Other methods of delivering energy may include but are not limited to other laser wavelengths, microwave, RF, and proton beam. The user (10) enters parameters into the energy device for a desired treatment. The energy device maintains these parameters and constantly monitors and controls the output energy.

The energy device (20) is able to track a time related treatment history (24) from information obtained from the light scattering device (30). This information includes a history of all previous results, rate of change of light scattering intensities as a result of temperature or tissue changes, algorithms to extrapolate future treatment effects based upon present and past data records. With this information, the energy device (20) will be capable of automatically controlling the delivery parameters to maintain temperature time information (24) programmed into the device by the user (14). The laser can adjust the power, interval, duration, intensity, and or duty cycle to create desired treatment effect rise time, duration at a given temperature effect, desired fluctuations over time, or desired decreases in treatment effects. This feature can be enabled or disabled by the user. Simultaneous to automatic control (26), the energy device (20) can inform the user (10) of the progress of the treatment through the use of a visual output (15), an audible output (16), or a printed output (17).

The scattered light/illumination device (30) has a diagnostic laser or illumination source (32) to view the retina being observed for temperature dependant changes. In addition, the measurement device (30) need not be separate from the energy delivery system (20). For example, the treatment beam itself, or aiming beam, could be used as the excitation beam (32) alleviating the need for an additional laser source. The incident light can be either polarized or non-polarized. If monitoring the effect of birefringence upon the eye, a system such as a scanning laser ophthalmoscope or phase sensitive optical coherence tomographer (PS-OCT) could be used. When using a PS-OCT there is the added benefit of being able to observe phase sensitive changes or changes in polarization at specific depths within the eye. An SLO or light source is capable of monitoring full thickness changes, but will also change as a result of tissue changes. In the case of thermal treatments where thermal elevation is highly localized through use of short irradiation times, phase sensitive measurements could be made in both the treatment location and in a neighboring section of tissue to provide increased detection sensitivity by comparing the two regions.

The delivery device (40) is used to image the energy from the energy device into a known spot size on the retina. The delivery device (40) can also be used to integrate the light scattering measurement device's excitation beam into the treatment energy's path. The delivery device (40) allows the user (10) to monitor the treatment progress while also combining all necessary aspects of the laser system.

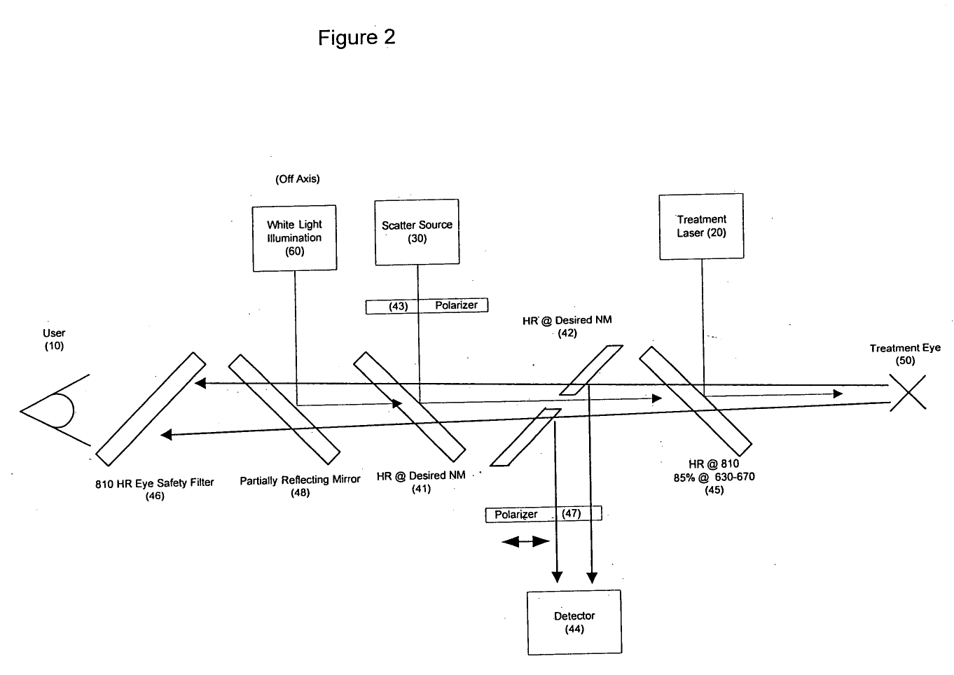

FIG. 2 shows an embodiment where the user (10) views the light through a slit lamp or other viewing mechanism to which the current invention attaches. In FIG. 2, the user (10) views the output of the delivery device that is lensed and focused in the slit lamp and delivered to the user (10). A safety filter (46) is positioned before the user (10) to block all treatment light from returning to the user's eye. This safety filter (46) can be a high reflector at the wavelength of the delivery laser and allows light outside that wavelength to pass.

Diagnostic illumination is provided to the treatment eye (50) from the White Light source (60) by a partially reflecting mirror (48). The mirror (48) is typically 50% reflective in the visible region and is usually part of the slit lamp viewing system. It can be delivered either on or off the viewing axis. Illuminating off axis allows the diagnostic device to function without interfering with visualization.

The scatter source (30) delivers an output excitation beam to scatter off the treatment eye (50). This output beam (scatter beam) passes through a polarizer (43) prior to being turned into the beam path by an optic (41) that is highly reflected at the scatter wavelength. This optic allows transmission of wavelengths other than the scatter beam wavelength and therefore does not affect visualization significantly. Once turned into the beam path, the scatter beam passes through a small hole in the center of mirror (42). The treatment laser is combined with the scatter beam through this mirror, which is highly reflective at the treatment laser wavelength. These two beams, and illumination light, are delivered co-linearly to the treatment eye (50).

Scattered light and reflected light from the treatment eye (50) is returned through optic (45). Most of the treatment beam is lost here as this optic is highly reflective to the treatment laser wavelength. The scattered light then reaches the optic, which is highly reflective at the scatter beam wavelength (42). A small amount of light will pass through the hole in the center of this optic but the scattered light in general is not collimated and the majority will reflect off the surface into another polarizer. This polarizer (47) is typically polarized at 90 degrees with respect to polarizer (43). (It could be an adjustable polarizer as in Iridex's TruView Product to allow the system to determine polarization and phase sensitive changes over 360 degrees.) The effect of the second polarizer is to remove all undesired reflected light and only allow scattered light relevant to the desired diagnostic method pass. This scattered light is then collected in the detector (44). The light picked up in the detector (44) is sent back to the light scattering device as data (34). The remaining light that was not reflected passes back to the first high reflector at the scatter wavelength. This blocks any additional light in that wavelength from reaching the operator's eye. The remaining light is partially reflected by mirror (48) and then passes through the eye safety filter, which removes any remaining treatment laser energy. The end view to the user is an unobstructed view of the retina illuminated by white light but missing a section of wavelengths at the treatment wavelength and at the scatter wavelength.

The user (10) can also adjust the treatment size on the retina by changing optics after the addition of the treatment laser (20). This is not required in a delivery device but increases the number of treatments that can be performed with a single device. Multiple delivery devices may also be used to provide various spot size selection and function with multiple ophthalmic treatment and viewing devices (i.e. various brands of slit lamps, LIOs, etc.) Information as to which spot size is selected is returned to the energy device (20) to allow for accurate power/intensity calculations and can be returned to the light scattering system (30) to provide any additional information if required regarding the excitation beam.

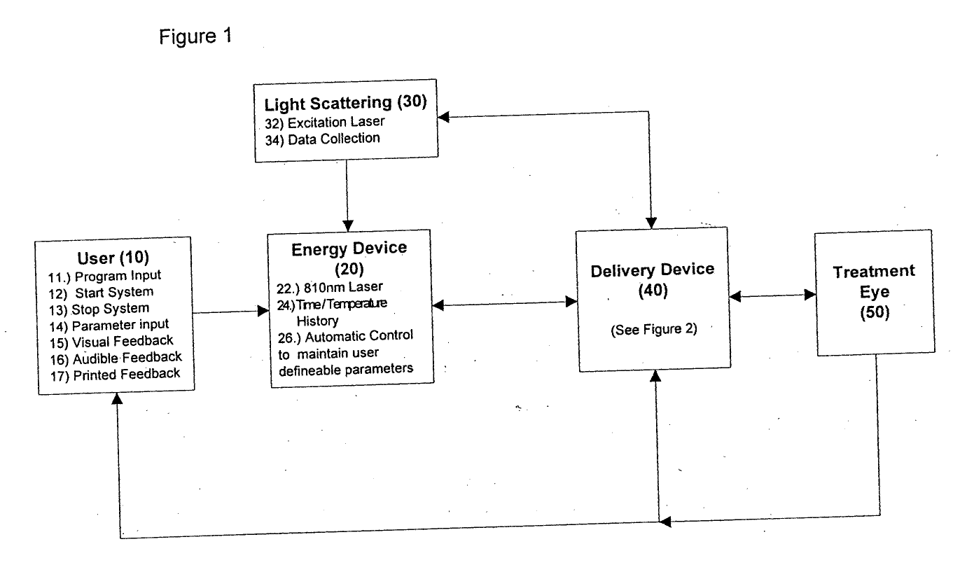

The system has been broken into discrete parts in FIG. 1 to diagram independent functions and is only one possible arrangement of the entire system. It is possible to combine multiple portions of the design to create a more user friendly and compact system. For example the processor can be a single processor used for the treatment laser, the light scattering measurement and to control the laser to maintain user defined temperature profiles. The light scattering excitation laser (32) could be the aiming beam for the treatment laser and the data collection (34) could be performed in the delivery device.

Changes in tissue can occur as direct thermal changes, or as changes induced by thermal energy but detected via chemical, mechanical, and/or optical changes. Mechanical changes can occur and manifest as physical changes. A mechanical change could be observed if an object changed location as a result of treatment. A detection method capable of monitoring scattered light at a certain depth in the tissue will observe a change in location as being a change in light scattering. Even though the scattering body need not change absolute scattering intensity, motion out of the monitoring volume will be detected. Chemical changes incurred by thermal treatment include but are not limited to protein denaturing, which is partially mechanical as well, and up-regulation of natural proteins and substances. A change in concentration of naturally occurring chemicals, if light scattering or birefringent, will result in monitored changes.

By way of illustration, and without limitation, during energy delivery to the eye, hemoglobin and other proteins, both in the retinal tissues and in choroidal and arterial blood, will begin to elevate in temperature. As they reach their denaturation point, some will begin to denature and their scattering intensity, primarily at the principal scattering wavelength, will begin to change. As the temperature rises, more proteins will denature further changing the scatter intensity. In the case of hemoglobin and other proteins carried by blood flow, the scatter intensity will be further temperature dependant. The blood will continuously carry normal proteins to the temperature-elevated region and remove denatured proteins. The proteins denatured as a result of temperature will only be present in the treatment area for as long as the flow rate allows. As the temperature increases, a larger percentage of proteins in the observation area will denature making the real time measured scattering changes temperature dependant. Maintaining a constant temperature induced change in scattering provides a method to deliver proper laser dosimetry to the eye.

Changes in scattering show magnitude of treatment effects on the retina. This is especially true in proteins that are not constantly refreshed by circulation. In these structures, scatter intensity changes will be dependant upon both the absolute temperature and the amount of time the region has been elevated. By monitoring the degree of scatter change in proteins of this nature, the absolute amount of damage created can be determined. Knowing the extent of a treatment and knowing the desired endpoint, provides the ability to terminate a treatment when a sufficient dosage has been delivered. This prevents the risk of over, or under exposure.

The ability to monitor temperature and it's affects on protein scattering provides many significant advantages to thermal procedures where the ability to monitor temperature directly is either difficult or impossible. In ophthalmology, laser treatments induces changes in the retina by creating thermal elevations of varying degree. The ability to monitor these changes real time increases the ability of a doctor to perform therapeutically effective and non-damaging treatments. TTT is just one such laser procedure that benefits from this. MicroPulse™ treatments are another laser treatment in ophthalmology that can benefit. Any sub-visible-threshold treatment in ophthalmology using non-invasive lasers can benefit from knowing either the temperature or the magnitude of effect of treatment on proteins in the eye. Retinal photocoagulation as well as thermal treatments on the sclera can benefit from information obtained from the tissue site.

Monitoring treatment-induced changes is beneficial in many areas of medicine. In dermatology, temperature measurements of the surface of the skin are taken to indirectly determine the proper dose of energy to provide skin rejuvenation through denaturing collagen without damaging the cellular structures. The ability of this system to directly monitor scattering from collagen would allow a device to provide sufficient energy to raise the temperature significantly enough to denature collagen while still enabling the system to protect the cellular structures. In the case of vascular lesions and hair removal, just the opposite is desired. Energy is absorbed at the tissue site but care is taken to minimize or prevent damage to collagen. The ability to detect damage to collagen provides an upper limit to energy delivery.

Collagen shrinkage is also used in ophthalmology for vision correction as described in U.S. Pat. No. 4,976,709, incorporated herein by reference. In this usage a desired intensity of treatment is used to shrink the collagen and in-turn, change the refraction of the cornea. The ability to detect the intensity of treatment can increase the ability to deliver optimum irradiation for vision correction and long-term stability.

In tumor treatments it is often desirable to damage vascular structures without damaging surrounding tissue (brain tumor as an example). This method would allow the user to deliver sufficient energy to denature proteins in the vascular system (hemoglobin, etc.) to a known level and thus prevent damage to other tissues with higher temperature thresholds. In addition, the ability to monitor changes in the structures desired not to change provides additional safety data to keep treatment temperatures below the damage threshold of the tissue that is being preserved.

By monitoring the back scattered light during a treatment, this method of measurement does not have any complications associated with self heating of a temperature measurement device as exists with conventional thermocouples and thermometers. With these methods, the treatment energy is partially absorbed in the temperature measurement device itself and can lead to false temperature measurements.

The foregoing description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

What is claimed is:

1. A method of monitoring a treatment site, comprising:
providing an apparatus for monitoring a temperature change at a tissue site, the apparatus including a scattered light measurement device that produces an excitation beam and an output device;
producing an excitation beam to scatter from the tissue site;
monitoring temperature or temperature changes of the tissue site; and
providing to an observer an indicative of the temperature change at the tissue site.

2. A method of monitoring a treatment site, comprising:
providing an apparatus for monitoring a temperature induced change at a tissue site, the apparatus including a scattered light measurement device that produces an excitation beam and an output device;
producing an excitation beam to scatter from the tissue site;
monitoring the temperature induced changes of the tissue site; and
providing to an observer an indicative of the temperature induced change at the tissue site.

3. An apparatus for monitoring a temperature change at a tissue site that is observed by an observer, comprising:
a scattered light measurement device that produces an excitation beam to scatter from the tissue site and monitor temperature or temperature changes of the tissue site; and
an output device that provides an output to the observer that is indicative of the temperature change at the tissue site.

4. The apparatus of claim 3, wherein the output is at least one of an output through a computer, an output through a heads up display, through a slit lamp, an audible output or a print out of information.

5. An apparatus for monitoring temperature induced changes at a tissue site, comprising:
a scattered light measurement device that produces an excitation beam to scatter from the tissue site and monitor temperature induced changes of the tissue site; and
an output device that provides an output to an observer that is indicative of the temperature induced change at the tissue site.

6. The apparatus of claim 5, wherein the output is at least one of an output through a computer, an output through a heads up display, through a slit lamp, an audible output or a print out of information.

7. A treatment apparatus for a tissue site, comprising:
an energy device that produces energy delivered to the tissue site;
a scattered light measurement device that delivers an excitation beam to scatter off the tissue site and monitor temperature, temperature changes or temperature induced changes of the tissue site; and
an output device that provides an output to an operator, which is indicative of the measured changes at the tissue site, such that the operator can control the treatment.

8. A treatment apparatus for a tissue site, comprising:
an energy device that produces energy delivered to the tissue site;
a scattered light measurement device that delivers an excitation beam to scatter off the treatment site and monitors temperature, temperature changes or temperature induced changes of the tissue site; and
a control device coupled to the energy device and the scattered light measurement device, which, in response to the changes, controls the output energy of the treatment beam to the tissue site.

9. The apparatus of claim 8, wherein the scattered light correlates to a birefringence effect resulting from the delivery of the treatment beam to the tissue site.

10. The apparatus of claim 8, wherein the scattered light correlates to a chemical effect resulting from the delivery of the treatment beam to the tissue site.

11. The apparatus of claim 8, wherein the scattered light correlates to a thermal effect resulting from the delivery of the treatment beam to the tissue site.

12. The apparatus of claim 8, wherein the scattered light correlates to a mechanical effect resulting from the delivery of the treatment beam to the tissue site.

13. The apparatus of claim 8, wherein the scattered light correlates to a polarization change resulting from the delivery of the treatment beam to the tissue site.

14. The apparatus of claim 8, wherein the treatment site is skin.

15. The apparatus of claim 8, wherein the treatment site is the cornea of an eye.

16. The apparatus of claim 8, wherein the treatment site is a tumor.

17. The apparatus of claim 8, wherein the treatment site is a vascular structure.

18. A treatment apparatus for an eye, comprising:
an energy device that produces a treatment beam delivered to a tissue site;
a scattered light measurement device that delivers an excitation beam to scatter off the treatment eye; and
a control device coupled to the energy device and the scattered light measurement device, in response to a change in the scattered light from the excitation beam, the control device controlling the output energy of the treatment beam while the scattered light measurement device monitors the change in scattered light.

19. The apparatus of claim 18, wherein the scattered light correlates to a birefringence effect resulting from the delivery of the treatment beam to the tissue site.

20. The apparatus of claim 18, wherein the scattered light correlates to a chemical effect resulting from the delivery of the treatment beam to the tissue site.

21. The apparatus of claim 18, wherein the scattered light correlates to a thermal effect resulting from the delivery of the treatment beam to the tissue site.

22. The apparatus of claim 18, wherein the scattered light correlates to a mechanical effect resulting from the delivery of the treatment beam to the tissue site.

23. The apparatus of claim 18, wherein the scattered light correlates to a polarization change resulting from the delivery of the treatment beam to the tissue site.

24. The apparatus of claim 18, wherein the treatment delivers the treatment beam to the tissue site until a threshold is reached.

25. The apparatus of claim 18, wherein the excitation beam of the scattered light measurement device is selected from a laser or an illumination source.

26. The apparatus of claim 18, wherein the scattered light measurement device is selected from a polarization device, a phase sensitive optical device, and a birefringent device.

27. The apparatus of claim 26, wherein the phase sensitive optical device is a phase sensitive optical coherence tomographer (PS-OCT).

28. The apparatus of claim 26, wherein the polarization device is a scanning laser ophthalmoscope.

29. The apparatus of claim 26, wherein the polarization device can vary polarization from 0 to 360 degrees.

30. The apparatus of claim 29, wherein the variation in polarization is of the source, the detector, or both.

31. The apparatus of claim 26, wherein the PS-OCT observes phase sensitive changes or changes in polarization at specific depths within the tissue site.

32. The apparatus of claim 26, wherein the polarization device monitors changes at variable and specific depths in the tissue site.

33. The apparatus of claim 26, wherein the polarization device monitors full thickness changes in the tissue site.

34. The apparatus of claim 18, wherein the scattered light measurement device provides measurements at the treatment site and at an off treatment site.

35. The apparatus of claim 18, wherein the scattered light measurement device provides measurement by comparing a current measurement to a baseline measurement at the treatment site.

36. The apparatus of claim 18, wherein the scattered light measurement device determines a change at the treatment site by comparing the off treatment site with the treatment site.

37. The apparatus of claim 18, wherein the measurements produce a map of the monitored area.

38. The apparatus of claim 18, wherein the scattered light measurement device measures absolute temperature.

39. The apparatus of claim 18, wherein the treatment beam has a wavelength that has sufficient transmission efficiency to pass through the cornea, lens and aqueous.

40. The apparatus of claim 18, wherein the treatment beam has a wavelength that is a visible or IR wavelength.

41. The apparatus of claim 18, wherein control device tracks a time related treatment history from information obtained from the scattered light measurement device.

42. The apparatus of claim 41, wherein the time related treatment history includes information selected from a history of all previous results, rate of change of scattered light intensities as a result of temperature or tissue changes, and algorithms to extrapolate future treatment based upon present and past data records.

43. The apparatus of claim 18, wherein the control device provides a signal to the energy delivery device to adjust a parameter of the energy device selected from power, interval, duration, intensity and duty cycle.

44. The apparatus of claim 18, wherein the parameter of the energy device is adjusted to create a desired treatment effect selected from rise time, duration at a given temperature effect, desired fluctuations over time and desired changes in treatment effects.

45. The apparatus of claim 18, wherein energy device and the scattered light measurement device are the same device.

46. The apparatus of claim 45, wherein the treatment beam is used as the excitation beam.

47. The apparatus of claim 18, further comprising:
a delivery device coupled to the light energy device.

48. The apparatus of claim 18, wherein the delivery device images the treatment beam from the light energy device into a known spot size on the retina.

49. The apparatus of claim 18, wherein the delivery device directs the excitation beam into a beam path of the treatment beam.

50. The apparatus of claim 18, further comprising:
a viewing device for viewing on axis.

51. The apparatus of claim 50, wherein the viewing device is a slit lamp.

52. The apparatus of claim 50, wherein the excitation beam is delivered off axis.

53. The apparatus of claim 18, further comprising:
an optical member that is highly reflective at the excitation wavelength.

54. The apparatus of claim 18, further comprising:
an optical device that provides a user with an unobstructed view of the eye illuminated by white light while missing a section of wavelengths at the treatment wavelength and at the excitation wavelength.

55. The apparatus of claim 19, wherein the tissue site is the optic nerve head.

56. The apparatus of claim 19, wherein the tissue site is Henle's layer.
